(19) United States
(12) Patent Application Publication
(10) Pub. No.: US 2026/0083326 A1
(43) Pub. Date: Mar. 26, 2026

(54) OPTICAL SENSOR SYSTEMS FOR MEASURING INTRAOCULAR PRESSURE

(71) Applicant: Alcon Inc., Fribourg (CH)

(72) Inventor: Steven T. Charles, Memphis, TN (US)

(21) Appl. No.: 19/332,767

(22) Filed: Sep. 18, 2025

Related U.S. Application Data
(60) Provisional application No. 63/698,150, filed on Sep. 24, 2024.

(51) Int. Cl. A61B 3/16

(52) U.S. Cl. CPC A61B 3/16

(57) ABSTRACT

An optical sensor system includes a light source and an optical fiber configured to transmit wavelengths of light received from the light source. A Bragg grating is disposed in the optical fiber. The Bragg grating is configured to reflect a wavelength of the wavelengths of light based on a force applied to the optical fiber that corresponds to an intraocular pressure (IOP). An optical spectrum analyzer is configured to detect the wavelength.

### INTRODUCTION

Maintaining intraocular pressure (IOP) within a desired range is an important factor during ophthalmic surgical interventions including, for example, refractive surgery, lens-replacement surgery, and retinal surgery. During surgical interventions, an ophthalmic surgical system uses infusion pressure to deliver an irrigation fluid into the interior of the eye, and the ophthalmic surgical system uses aspiration (vacuum) pressure to remove vitreous and lens fragments from the interior of the eye. The infusion pressure and the aspiration pressure of the ophthalmic surgical system are adjusted to control the IOP.

### SUMMARY

Aspects of the present disclosure relate to measuring intraocular pressure (IOP), and more specifically, to optical sensors systems for measuring IOP.

In certain embodiments, an optical sensor system includes a light source and an optical fiber configured to transmit wavelengths of light received from the light source. A Bragg grating is disposed in the optical fiber. The Bragg grating is configured to reflect a first wavelength of the wavelengths of light based on a first force applied to the optical fiber that corresponds to a first intraocular pressure (IOP). An optical spectrum analyzer is configured to detect the first wavelength.

In certain embodiments, a method includes transmitting wavelengths of light through an optical fiber that includes a Bragg grating. The method further includes reflecting, by the Bragg grating, a first wavelength of the wavelengths of light based on a first force applied to the optical fiber that corresponds to a first intraocular pressure (IOP). The method further includes detecting the first wavelength. The method further includes determining the first IOP based on the first wavelength.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates a representation of a human eye, according to embodiments described herein.

FIGS. 2A and 2B illustrate a portion of an optical fiber that includes a Bragg grating, according to embodiments described herein.

FIG. 3 illustrates a representation of a Bragg grating disposed in a portion of an infusion cannula, according to embodiments described herein.

FIG. 4 illustrates a representation of a Bragg grating disposed in a portion of a phacoemulsification handpiece, according to embodiments described herein.

FIG. 5 illustrates an example of a surgical system, according to embodiments described herein.

FIG. 6 illustrates an example method for measuring intraocular pressure (IOP), according to embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

Aspects of the present disclosure relate to intraocular visualization, and more specifically, to calibrating intraocular illumination light transmitted from a light source.

The designations “first” and “second” as used herein are not meant to indicate or imply any particular positioning or other characteristic. Rather, when the designations “first” and “second” are used herein, they are used only to distinguish one component from another. The terms “attached,” “connected,” “coupled,” and the like mean attachment, connection, coupling, etc., of one part to another either directly or indirectly through one or more other parts, unless direct or indirect attachment, connection, coupling, etc., is specified.

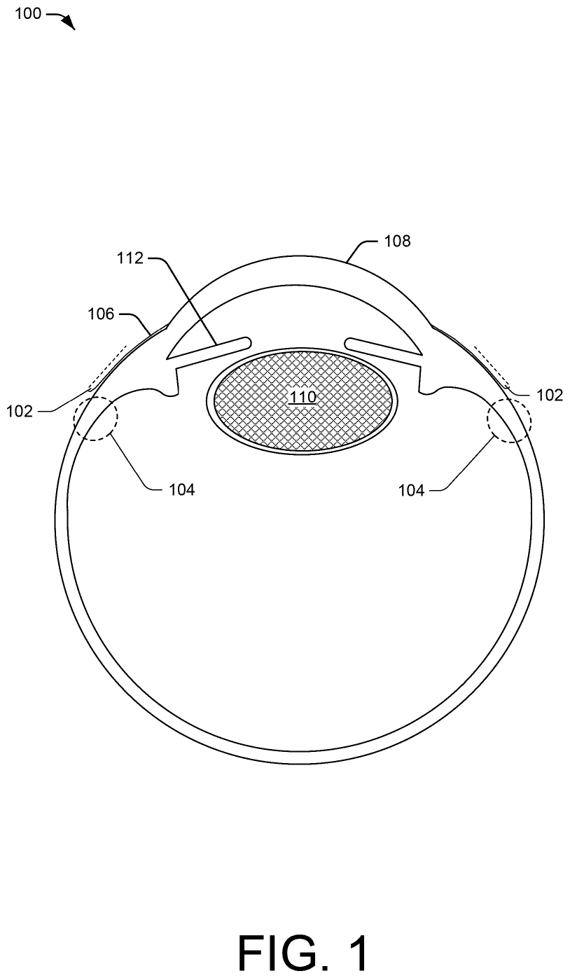

FIG. 1 illustrates a representation of a human eye 100, according to embodiments described herein. As depicted in FIG. 1, the representation illustrates a fornix 102, a pars plana 104, a sclera 106, a cornea 108, a lens 110, and an iris 112. The fornix 102 is a recessed portion of the conjunctiva that is formed where the eyelids interface with the sclera 106.

The pars plana 104 is a region within the ciliary body commonly utilized to access the posterior segment during vitreoretinal surgical procedures (e.g., to remove portions of the vitreous humor). This access is typically achieved via cannulas which are inserted into small incisions made in the pars plana 104 during the procedure. For instance, the cannulas may include valves for maintaining the intraocular pressure (IOP) of the eye 100. Controlling the IOP is important because high IOP (e.g., ocular hypertension) increases the risk of developing arterial occlusion, ischemic damage and eventually glaucoma and low IOP (e.g., ocular hypotony) can reduce structural support for the eye 100 resulting in bleeding, pupil constriction, ocular collapse and eventually vision loss.

The sclera 106 is the white/opaque fibrous tissue that is the structural layer of the outer eye and forms its round shape when the IOP is within a normal range. The sclera 106 extends from the cornea 108 to the optic nerve at the back of the eye 100. The cornea 108 covers the iris 112 which is the colored part of the eye 100 that controls the size of the pupil. The pupil allows light into the eye 100 which the lens 110 focuses on the retina at the back of the eye 100. The retina includes photoreceptor cells which convert the light into signals for visual perception. Notably, high IOP can interrupt blood flow to the retina and the optic nerve which may result in damage to the optic nerve and diminished visual perception.

In order to periodically monitor and measure the IOP during surgical interventions, various types of pressure sensors have been conventionally utilized. These periodic IOP measurements are used as a basis for adjusting infusion or aspiration pressure supplied to the eye 100 by an ophthalmic surgical system to maintain the IOP at the desired level. To measure the IOP, the pressure sensors are incorporated into instruments such as phacoemulsification handpieces. For example, a microelectromechanical system (MEMS) sensor is disposed within the handpiece adjacent to an aspiration line. The MEMS sensor measures forces at the aspiration line which can be used to estimate an IOP within the eye 100. However, MEMS sensors are associated with several disadvantages such as being relatively large in size and having a relatively high manufacturing cost. MEMS sensors can also be damaged during cleaning/sterilization of phacoemulsification handpieces before or after surgical procedures.

Accordingly, certain embodiments herein provide optical sensor systems including an optical fiber, a light source, and an optical spectrum analyzer for measuring IOP. The optical sensor systems described herein measure a force of the IOP by transmitting light through the optical fiber via the light source such that the force of the IOP is applied to the optical fiber. As the light is transmitted through the optical fiber, the force applied to the optical fiber causes a change in the transmission of the light that uniquely corresponds to the force. The change in the transmission of the light is detected using the optical spectrum analyzer, and the detected change in the transmission of the light is mapped to the force of the IOP.

For example, in some embodiments, an optical fiber of an optical sensor system provided herein may include a Bragg grating that is configured to reflect a first wavelength of light and transmit all other wavelengths of light. When a force is applied to the Bragg grating, the Bragg grating deforms which causes the Bragg grating to reflect a second wavelength of light instead of the first wavelength. The second wavelength corresponds to the force applied to the Bragg grating. Therefore, by detecting the second wavelength, the force applied to the Bragg grating can be determined.

The optical sensor system described herein do not have the corresponding disadvantages of MEMS sensors. For example, the optical sensor systems herein are small and durable enough to be sterilized in a medical autoclave and have low enough manufacturing costs to be included as part of a single-use instrument/device. Compared to conventional MEMS sensors, the optical sensor systems described herein are more robust to heat exposure and handling during cleaning or sterilization procedures. The optical sensor systems are also more resistant to saline than the MEMS sensors which can be damaged by electrochemical corrosion (e.g., from extended exposure to sodium chloride).

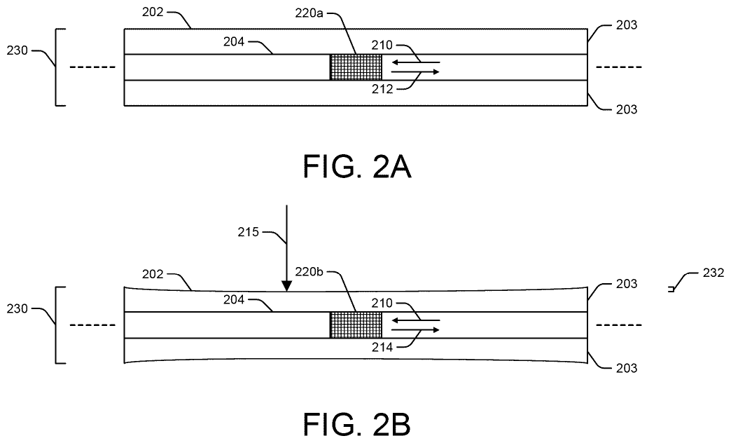

FIGS. 2A and 2B illustrate a portion of an optical fiber 202 that includes a Bragg grating 220a, according to embodiments described herein. FIG. 2A illustrates a schematic cross-sectional view of the portion of the optical fiber 202. The optical fiber 202 includes a cladding 203 and a core 204 disposed in the cladding 203. An outer diameter 230 of the optical fiber 202 is illustrated to include the cladding 203 and the core 204. In some embodiments, the outer diameter 230 is less than or equal to 50 micrometers (μm). Because of the relatively small size of the outer diameter 230, the optical fiber 202 can be incorporated into a variety of different devices and instruments as described below.

A Bragg grating 220a is formed in the core 204 of the optical fiber 202. The Bragg grating 220a is a segment of the core 204 with a grating pattern that causes a periodic variation in the refractive index of the optical fiber 202. In some embodiments, the Bragg grating 220a is inscribed in the core 204 using an ultraviolent wavelength laser that is typically masked or split to inscribe the grating pattern in the core 204. The periodic variation in the refractive index reflects light at a particular wavelength and transmits light at all other wavelengths. As shown in FIG. 2A, wavelengths of light 210 are transmitted through the core 204 of the optical fiber 202 and the Bragg grating 220a reflects a first wavelength 212 of the wavelengths of light 210.

FIG. 2B illustrates a force 215 being applied to the portion of the optical fiber 202. When the force 215 is applied to the optical fiber 202, the force 215 causes the optical fiber 202 (and the Bragg grating 220a) to deform such that a portion of the outer diameter 230 is compressed by a compressed distance 232. Deformation of the optical fiber 202 (e.g., by the compressed distance 232) changes spacing in the grating pattern of the Bragg grating 220a and a deformed Bragg grating 220b shifts the wavelength of light reflected from the first wavelength 212 to a second wavelength 214.

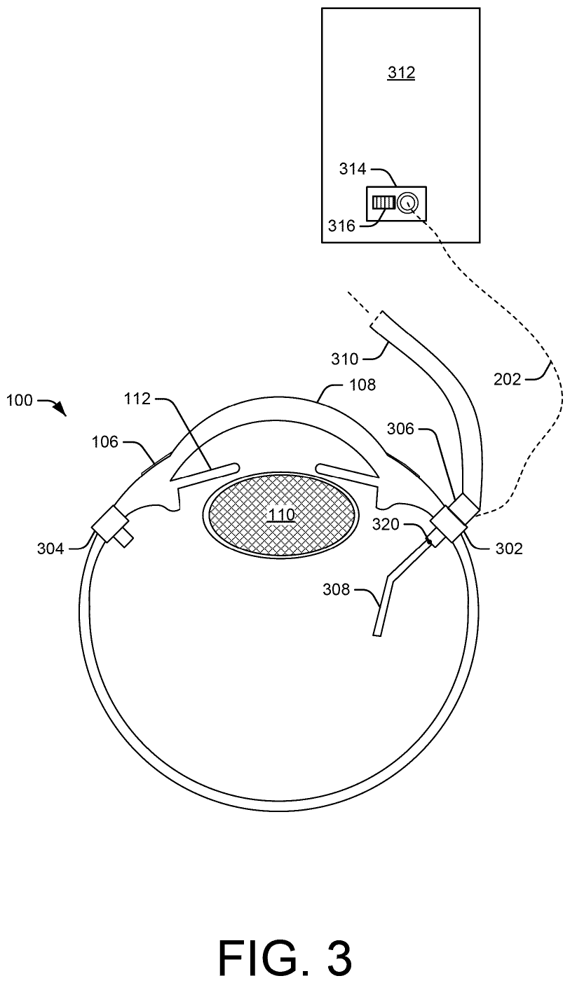

As shown in FIG. 2B, the wavelengths of light 210 are transmitted through the core 204 of the optical fiber 202 and the Bragg grating 220b reflects the second wavelength 214 of the wavelengths of light 210 based on the force 215 applied to the optical fiber 202. An optical spectrum analyzer (e.g., optical spectrum analyzer 316, shown in FIG. 3) then detects the second wavelength 214 as being reflected when the force 215 is applied to the optical fiber 202 (e.g., the second wavelength 214 is included in a spectrum of reflected light received by the optical spectrum analyzer). In this example, since the second wavelength 214 corresponds to the force 215, the force 215 can be determined (e.g., indirectly measured) and an IOP that is associated with the force 215 may be identified as described below.

Although the optical fiber 202 is illustrated and described to include the Bragg grating 220a, it is to be appreciated that, in some embodiments, the optical fiber 202 may include one or more other types of optical sensors. For example, the optical fiber 202 can include interferometric sensors, phase-based sensors, intensity-based sensors, polarimetric sensors, and other sensors. Similarly, while the optical fiber 202 is shown as including one optical sensor (e.g., the Bragg grating 220a), in some embodiments, the optical fiber 202 includes multiple sensors such as multiple Bragg gratings 220a or multiple sensors of different types. Notably, the optical fiber 202 and the Bragg grating 220a are not limited to measuring pressure within a particular physical medium such as balanced salt solution (BSS) used in some ophthalmic surgical interventions. Rather, the optical fiber 202 and the Bragg grating 220a are capable of measuring pressure within saline, BSS, silicone oil, air/gas, and/or the like.

FIG. 3 illustrates a representation of a Bragg grating 320 disposed in a portion of an infusion cannula 306, according to embodiments described herein. As shown, the eye 100 includes cannulas 302, 304 inserted into small incisions made in the pars plana 104. The infusion cannula 306 is inserted into the cannula 302 for delivering silicone oil into the vitreous cavity. The silicone oil is disposed in an infusion tube 310 and then delivered into the vitreous cavity via a hypodermic tube 308. Once delivered, the silicone oil acts as an internal tamponade to support the retina.

As the silicone oil is being delivered into the vitreous cavity, numerous events may occur or fail to occur which can cause the IOP within the eye 100 to increase or decrease. For example, inserting the infusion cannula 306 into the cannula 302 could indent the sclera 106 causing the IOP to increase, removing the infusion cannula 306 from the cannula 302 might inadvertently pull the cannula 302 out from the incision in the pars plana 104 causing the IOP to decrease, or some other event could occur or fail to occur causing the IOP to increase or decrease. In order to measure the IOP within the eye 100 and identify any such increases or decreases, the optical fiber 202 described in relation to FIG. 2A is included in an optical sensor system that also includes a light source 312 and an optical spectrum analyzer 316. The optical fiber 202 includes the Bragg grating 320 and the optical fiber 202 is disposed in an optical interface 314 of the light source 312 such that the core 204 receives the wavelengths of light 210 from the light source 312 and the wavelengths of light 210 are transmitted through the optical fiber 202. In some embodiments, the light source 312 and the optical spectrum analyzer 316 are included in a surgical system which also includes an infusion and aspiration subsystem that controls an infusion pressure delivered to the infusion tube 310. In other embodiments, the light source 312 and the optical spectrum analyzer 316 are part of a standalone system or are included in another device or system.

The optical fiber 202 receives the wavelengths of light 210 from the light source 312 (e.g., via the optical interface 314) and transmits the wavelengths of light 210 to the Bragg grating 320. In FIG. 3, the Bragg grating 320 is disposed in the hypodermic tube 308 and the IOP of the eye 100 causes a force (e.g., the force 215 shown in FIG. 2B) to be applied to the optical fiber 202 and the Bragg grating 320. The applied force deforms the optical fiber 202 and the Bragg grating 320 which causes the Bragg grating 320 to reflect the second wavelength 214 of the wavelengths of light 210. In some embodiments, the optical spectrum analyzer 316 detects the reflected second wavelength 214.

As described above, each of the reflected wavelengths of light 210 corresponds to a particular force applied to the optical fiber 202 and each particular force applied to the optical fiber 202 is associated with an IOP of the eye 100. In some embodiments, one or more processors of the optical spectrum analyzer 316, the light source 312, or the surgical system execute instructions that cause the one or more processors to access correlation data that maps each particular wavelength included in the wavelengths of light 210 to a corresponding force/IOP. For example, if the optical spectrum analyzer 316 detects the second wavelength 214, then a corresponding IOP can be identified (e.g., indirectly measured) using the correlation data.

If the IOP of the eye 100 has increased or decreased to a level that is outside of a desired range, then a user may be alerted to the increased or decreased IOP (e.g., an indication of the IOP may be output to alert the user). Additionally or alternatively, a pressure setting (e.g., infusion or aspiration) of a surgical system may be automatically adjusted to maintain the IOP of the eye 100 within the desired range. For example, if the Bragg grating 320 previously reflected the first wavelength 212 corresponding to a first IOP within the desired range, and subsequently the optical spectrum analyzer 316 detects a second wavelength 214 corresponding to a second IOP that is outside of the desired range, then one or more processors of the surgical system execute instructions which cause an infusion and aspiration subsystem to modify the pressure setting (e.g., infusion or aspiration) based on a difference between the first IOP and the second IOP.

Although the Bragg grating 320 is disposed in the hypodermic tube 308 in the example of FIG. 3, it is to be appreciated that, in some embodiments, the Bragg grating 320 can be disposed in a different portion of the hypodermic tube 308, another location in the infusion cannula 306, the infusion tube 310, or in a different location where the IOP of the eye 100 causes the force to be applied to the optical fiber 202 and the Bragg grating 320. While the optical fiber 202 and the Bragg grating 320 are described to be included in the infusion cannula 306, it is to be appreciated that, in various embodiments, the optical fiber 202 and the Bragg grating 320 can be included in another cannula such as a sclerotomy cannula, in a device such as a vitreous cutter, and/or in another instrument or device. Further, it is to be appreciated that a pressure sensor of the infusion and aspiration subsystem is separated from the eye 100 by a substantial amount of the infusion tube 310 (e.g., about 72 inches of tubing) which has inherent fluidic resistance. However, when the optical fiber 202 and the Bragg grating 320 are included in, for example, the infusion cannula 306, the described optical sensor systems are capable of measurements at the eye 100 for accurately measuring pressures in substantially real-time.

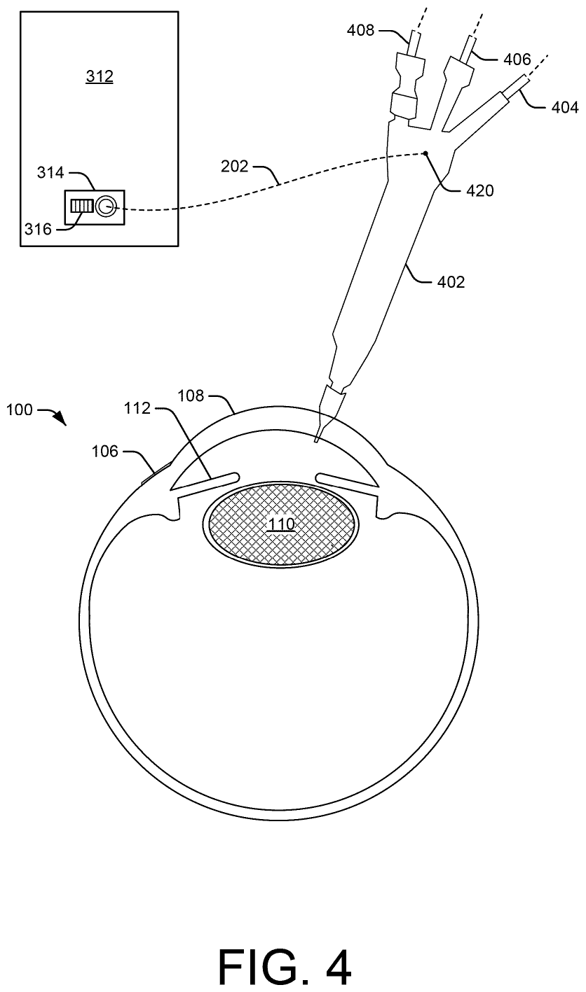

FIG. 4 illustrates a representation of a Bragg grating 420 disposed in a portion of a phacoemulsification handpiece 402, according to embodiments described herein. The phacoemulsification handpiece 402 is being used to fragment a cataractous lens in an anterior segment procedure. During the procedure, numerous events may occur or fail to occur which can cause the IOP within the eye 100 to increase or decrease. The phacoemulsification handpiece 402 includes a cable 404, an aspiration (vacuum) line 406, and an infusion (irrigation) line 408.

The optical fiber 202 is disposed in the optical interface 314 of the light source 312 and the optical fiber 202 includes the Bragg grating 420. In some embodiments, the optical fiber 202 receives the wavelengths of light 210 from the light source 312 and transmits the wavelengths of light 210 to the Bragg grating 420. As shown in FIG. 4, the Bragg grating 420 is disposed within a housing of the phacoemulsification handpiece 402. In some embodiments, the Bragg grating 420 is disposed within or adjacent to the aspiration line 406 of the phacoemulsification handpiece 402.

Similar to the example described in relation to FIG. 3, the IOP within the eye 100 causes a force to be applied to the optical fiber 202 that deforms the optical fiber 202 and the Bragg grating 420. The deformed Bragg grating 420 reflects a particular wavelength of the wavelengths of light 210 and the optical spectrum analyzer 316 detects the particular wavelength. One or more processors of the optical spectrum analyzer 316, the light source 312, or the surgical system execute instructions that cause the one or more processors to access the correlation data to identify (e.g., indirectly measure) a particular IOP corresponding to the particular wavelength. An indication of the particular IOP may be output to alert the user or a pressure setting (e.g., infusion or aspiration) can be automatically adjusted based on the particular IOP as described above.

Although the optical fiber 202 and the Bragg grating 420 are described to be included in the phacoemulsification handpiece 402, it is to be appreciated that, in various embodiments, the optical fiber 202 and the Bragg grating 420 can be included in a fragmentation handpiece, an irrigation and aspiration handpiece, and/or in another instrument or device. In some embodiments, the Bragg grating 420 can be disposed within or adjacent to the infusion line 408. Notably, the Bragg grating 420 can be disposed in any location where the IOP of the eye 100 causes forces to be applied to the optical fiber 202 and the Bragg grating 420. In some embodiments, the optical fiber 202 and the Bragg grating 420 are sterilized in a medical autoclave before use in the anterior segment procedure (e.g., before the Bragg grating 420 reflects the particular wavelength). The optical fiber 202 and the Bragg grating 420 can be sterilized in the medical autoclave multiple times without degradation such as 5 times, 10 times, or more than 10 times. Accordingly, the optical fiber 202 can be included in a reusable medical device component or a single-use medical device component.

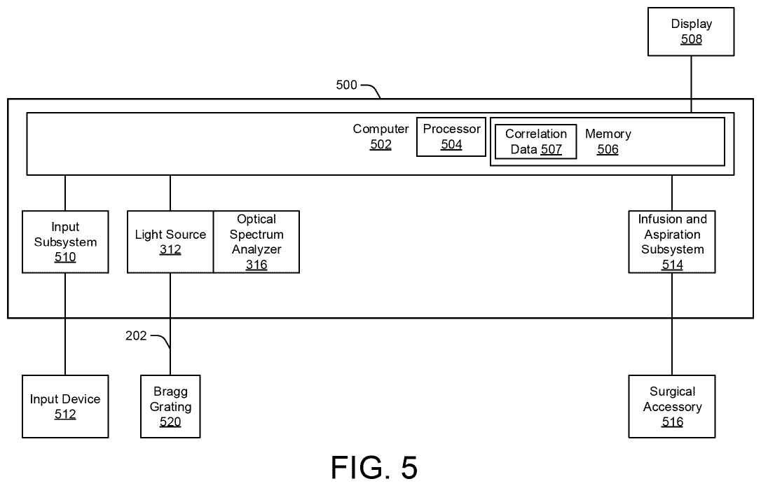

FIG. 5 illustrates an example of a surgical system 500, according to embodiments described herein. In some embodiments, the surgical system 500 includes an ophthalmic surgical console for performing ophthalmic surgical procedures. The surgical system 500 includes a computer 502 with a processor 504 (e.g., one or more processors) and a memory 506. In some embodiments, the memory 506 includes correlation data 507 that maps particular wavelengths of light to corresponding IOPs. For example, the correlation data 507 describes information that maps various wavelengths to various IOPs. A display device 508 is coupled to the computer 502. The display device 508 can include a user interface configured to receive user inputs.

The surgical system 500 includes an input subsystem 510 that is coupled to the computer 502 and an input device 512 such as a footswitch. In the example of FIG. 5, the surgical system 500 includes the light source 312 and the optical spectrum analyzer 316. The light source 312 is coupled to the computer 502. The light source 312 is illustrated to be optically coupled to a Bragg grating 520 (e.g., via the optical fiber 202). The Bragg grating 520 is incorporated into a surgical accessory 516 (e.g., infusion cannula 306, phacoemulsification handpiece 402, etc.). The surgical accessory 516 is controlled at least by an infusion and aspiration subsystem 514, or one or more other components of the surgical system 500, depending on the functionality of the surgical accessory 516.

In some embodiments, the light source 312 transmits light through the optical fiber 202. An IOP of the eye 100 applies a force to the optical fiber 202 and the Bragg grating 520. The force of the IOP deforms the Bragg grating 520 which causes the Bragg grating 520 to reflect the second wavelength 214 of the light. The optical spectrum analyzer 316 detects the second wavelength 214. The processor 504 (e.g., the one or more processors) accesses the correlation data 507 and identifies the IOP of the eye 100 based on the second wavelength 214.

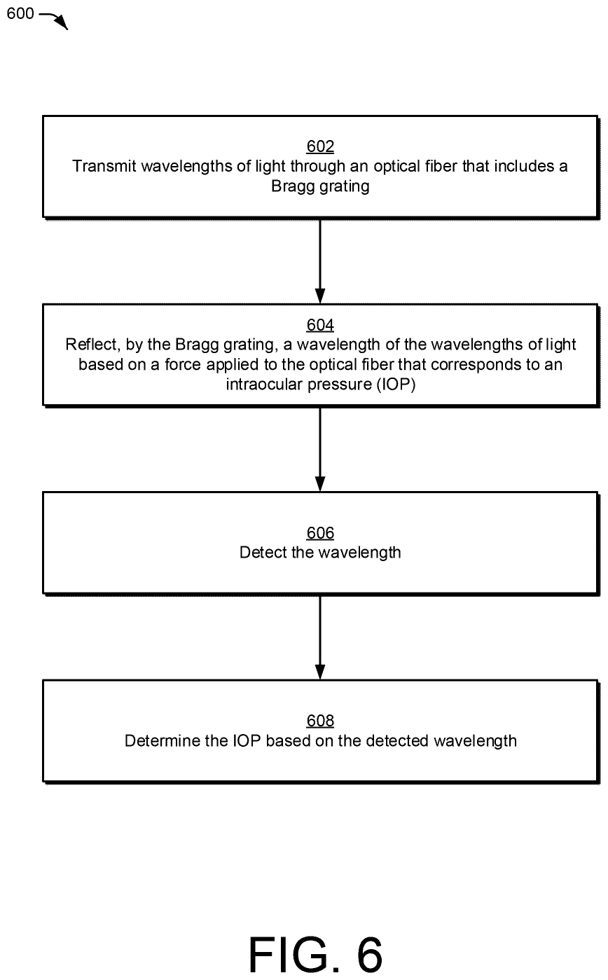

FIG. 6 illustrates an example method 600 for measuring intraocular pressure (IOP), according to embodiments described herein. At 602, wavelengths of light are transmitted through an optical fiber that includes a Bragg grating. In some embodiments, the wavelengths of light 210 are received from the light source 312 and the wavelengths of light 210 are transmitted through the core 204 to the Bragg grating 220.

At 604, a wavelength of the wavelengths of light (e.g., second wavelength 214) is reflected by the Bragg grating based on a force applied to the optical fiber that corresponds to an IOP.

At 606, the wavelength is detected. In some embodiments, the optical spectrum analyzer 316 detects the wavelength.

At 608, the IOP is determined based on the detected wavelength. In some embodiments, the IOP is determined (indirectly measured) by one or more processors based on detecting the wavelength by correlating the detected wavelength to the IOP using, for example, correlation data (e.g., in the correlation data 507).

The disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description.

What is claimed is:

1. An optical sensor system comprising:
a light source;
an optical fiber configured to transmit wavelengths of light received from the light source;
a Bragg grating disposed in the optical fiber, wherein the Bragg grating is configured to reflect a wavelength of the wavelengths of light based on a force applied to the optical fiber that corresponds to an intraocular pressure (IOP); and
an optical spectrum analyzer configured to detect the wavelength.

2. The optical sensor system of claim 1, further comprising one or more processors configured to execute instructions that cause the one or more processors to determine the IOP based on the detected wavelength.

3. The optical sensor system of claim 2, wherein the one or more processors are configured to determine the IOP using correlation data that maps various wavelengths to various IOPs.

4. The optical sensor system of claim 2, wherein the one or more processors are configured to output an indication of the IOP.

5. The optical sensor system of claim 2, wherein the one or more processors are configured to cause an infusion or aspiration subsystem to adjust an infusion pressure setting or an aspiration pressure setting, respectively, based on the IOP.

6. The optical sensor system of claim 1, wherein the Bragg grating is disposed in a portion of a phacoemulsification handpiece.

7. The optical sensor system of claim 1, wherein the Bragg grating is disposed in a portion of an infusion cannula.

8. The optical sensor system of claim 1, wherein the optical fiber has an outer diameter less than or equal to 50 micrometers.

9. A method comprising:
transmitting wavelengths of light through an optical fiber that includes a Bragg grating;
reflecting, by the Bragg grating, a wavelength of the wavelengths of light based on a force applied to the optical fiber that corresponds to an intraocular pressure (IOP);
detecting the wavelength; and
determining the IOP based on the detected wavelength.

10. The method of claim 9, further comprising modifying an infusion pressure setting or an aspiration pressure setting based on the IOP.

11. The method of claim 9, wherein the Bragg grating is disposed in a portion of a phacoemulsification handpiece.

12. The method of claim 9, wherein the Bragg grating is disposed in a portion of an infusion cannula.

13. The method of claim 9, wherein the optical fiber has an outer diameter less than or equal to 50 micrometers.

14. The method of claim 9, further comprising determining the IOP based on the detected wavelength.

15. The method of claim 14, wherein determining the IOP includes using correlation data that maps various wavelengths to various IOPs.
